SEQUENCE LISTING (43 sequences)

SEQ ID NO: 1 - DNA, 65 bp, Artificial Sequence
  Nucleotide sequence of miR-155-5p
ctgttaatgctaatcgtgataggggtttttgcctccaactgactcctacatattagcattaacag

SEQ ID NO: 2 - DNA, 19 bp, Artificial Sequence
  Upstream primer for GAPDH
aatcgccgtacccctacga

SEQ ID NO: 3 - DNA, 19 bp, Artificial Sequence
  Downstream primer for GAPDH
gccctatatgagctcctgt

SEQ ID NO: 4 - DNA, 18 bp, Artificial Sequence
  Upstream primer for miR-320a
tacccgttggctccaggt

SEQ ID NO: 5 - DNA, 20 bp, Artificial Sequence
  Downstream primer for miR-320a
ccggtggttttgtgacatcc

SEQ ID NO: 6 - DNA, 18 bp, Artificial Sequence
  Upstream primer for miR-146-5p
gctaagcttactggttag

SEQ ID NO: 7 - DNA, 20 bp, Artificial Sequence
  Downstream primer for miR-146-5p
ccctcggtatttacgccggt

SEQ ID NO: 8 - DNA, 18 bp, Artificial Sequence
  Upstream primer for miR-372
gactaagcgcctttcgta

SEQ ID NO: 9 - DNA, 20 bp, Artificial Sequence
  Downstream primer for miR-372
aacgatggacgcccgtatcc

SEQ ID NO: 10 - DNA, 19 bp, Artificial Sequence
  Upstream primer for miR-276-3p
atgcctcgttcatcaagaa

SEQ ID NO: 11 - DNA, 20 bp, Artificial Sequence
  Downstream primer for miR-276-3p
tagtcccctaacacattaag

SEQ ID NO: 12 - DNA, 19 bp, Artificial Sequence
  Upstream primer for miR-7a
attgccccccgcaggacct

SEQ ID NO: 13 - DNA, 20 bp, Artificial Sequence
  Downstream primer for miR-7a
tgcgcttgaaacccgccagg

SEQ ID NO: 14 - DNA, 19 bp, Artificial Sequence
  Upstream primer for miR-155-5p
aattttgacccgcaaggcc

SEQ ID NO: 15 - DNA, 21 bp, Artificial Sequence
  Downstream primer for miR-155-5p
tccaacgtagctgtcctgctt

SEQ ID NO: 16 - DNA, 20 bp, Artificial Sequence
  Upstream primer for miR-280-3p
aaaagttcccgccctgtata

SEQ ID NO: 17 - DNA, 21 bp, Artificial Sequence
  Downstream primer for miR-280-3p
tgggctagcacccttcggact

SEQ ID NO: 18 - DNA, 20 bp, Artificial Sequence
  Upstream primer for miR-451
cccccgaggttgccgaaatt

SEQ ID NO: 19 - DNA, 21 bp, Artificial Sequence
  Downstream primer formiR-451
ttgaacaaattggtcccttac

SEQ ID NO: 20 - DNA, 21 bp, Artificial Sequence
  Upstream primer for miR-220a
ccctccctaaggttatttgca

SEQ ID NO: 21 - DNA, 21 bp, Artificial Sequence
  Downstream primer for miR-220a
aatccggctcacccaattctg

SEQ ID NO: 22 - DNA, 20 bp, Artificial Sequence
  Upstream primer for miR-483-5p
gttccagcccggcacaatcg

SEQ ID NO: 23 - DNA, 21 bp, Artificial Sequence
  Downstream primer for miR-483-5p
taagacgctttcccggcgtgt

SEQ ID NO: 24 - DNA, 20 bp, Artificial Sequence
  Upstream primer for miR-144-3p
gacccgtggcttaactgcca

SEQ ID NO: 25 - DNA, 21 bp, Artificial Sequence
  Downstream primer for miR-144-3p
tgggtcctggccccggatttc

SEQ ID NO: 26 - DNA, 20 bp, Artificial Sequence
  Upstream primer for miR-26a
taatacaggccgggtcaaga

SEQ ID NO: 27 - DNA, 21 bp, Artificial Sequence
  Downstream primer for miR-26a
acggttcggtacccctatacg

SEQ ID NO: 28 - DNA, 21 bp, Artificial Sequence
  Upstream primer for miR-223
gagactactgctgtgggtaag

SEQ ID NO: 29 - DNA, 21 bp, Artificial Sequence
  Downstream primer for miR-223
cctttgttcttaacccagtga

SEQ ID NO: 30 - DNA, 21 bp, Artificial Sequence
  Upstream primer for miR-124
ctcccaacggtggcctcaatg

SEQ ID NO: 31 - DNA, 21 bp, Artificial Sequence
  Downstream primer for miR-124
tggccagaattccctgcagta

SEQ ID NO: 32 - DNA, 21 bp, Artificial Sequence
  Upstream primer for miR-123-5p
tgggccacatcccggctgcga

SEQ ID NO: 33 - DNA, 21 bp, Artificial Sequence
  Downstream primer for miR-123-5p
acccgtgaactggccagccta

SEQ ID NO: 34 - DNA, 21 bp, Artificial Sequence
  Upstream primer for miR-206
tgttgcacccccagagcattg

SEQ ID NO: 35 - DNA, 21 bp, Artificial Sequence
  Downstream primer for miR-206
aaagctccccggacgttagta

SEQ ID NO: 36 - DNA, 21 bp, Artificial Sequence
  Upstream primer for miR-21-5p
agaccggggaattgtgaggcc

SEQ ID NO: 37 - DNA, 21 bp, Artificial Sequence
  Downstream primer for miR-21-5p
tccttaaaggcaccctcctgg

SEQ ID NO: 38 - DNA, 21 bp, Artificial Sequence
  Upstream primer for miR-381-3p
ttgcggccgtcccttacgatc

SEQ ID NO: 39 - DNA, 21 bp, Artificial Sequence
  Downstream primer for miR-381-3p
cgtgccctgggcgtggtaccg

SEQ ID NO: 40 - DNA, 21 bp, Artificial Sequence
  Upstream primer for miR-340a
gttaagtgcgttaagacccgt

SEQ ID NO: 41 - DNA, 22 bp, Artificial Sequence
  Downstream primer for miR-340a
cttgcccaactcctccgtaatg

SEQ ID NO: 42 - DNA, 21 bp, Artificial Sequence
  Upstream primer for miR-132-3p
tcggtaacccgtgcgtgatta

SEQ ID NO: 43 - DNA, 21 bp, Artificial Sequence
  Downstream primer for miR-132-3p
cggttgccttcaagcctgaac

(19) United States
(12) Patent Application Publication
(10) Pub. No.: US 2022/0062346 A1
(43) Pub. Date: Mar. 3, 2022

(54) USE OF miRNA IN PREPARATION OF A DRUG FOR PREVENTING AND TREATING OSTEOARTHRITIS, AN EXOSOME HIGHLY EXPRESSING miRNA AND USE THEREOF

(71) Applicant: SUZHOU MUNICIPAL HOSPITAL (NORTH DISTRICT), Suzhou City (CN)

(72) Inventors: Yuefeng Hao, Suzhou City (CN); Xing Yang, Suzhou City (CN); Jing Zhou, Suzhou City (CN); Menglei Xu, Suzhou City (CN); Dechun Geng, Suzhou City (CN)

(73) Assignee: SUZHOU MUNICIPAL HOSPITAL (NORTH DISTRICT), Suzhou City (CN)

(21) Appl. No.: 17/347,867

(22) Filed: Jun. 15, 2021

(30) Foreign Application Priority Data
Aug. 25, 2020 (CN) 202010863064.X

(51) Int. Cl. A61K 35/28; A61P 19/02; C12N 5/0775; C12N 15/113

(52) U.S. Cl. CPC A61K 35/28; A61P 19/02; C12N 2320/32; C12N 15/113; C12N 2310/141; C12N 5/0662

(57) ABSTRACT

The present disclosure relates to the field of miRNA and exosomes, in particular to the use of miRNA in preparation of drugs for preventing and treating osteoarthritis, and the use of miRNA exosomes with high expression. The present disclosure provides the use of miR-155-5p in preparation of drugs for preventing and/or treating osteoarthritis. The nucleotide sequence of miR-155-5p is set forth in SEQ ID NO.:1. Use of the present disclosure can improve miR-155-5p expression by targeting miR-155-5p to Runx2, promote chondrocyte growth proliferation, migration, and extracellular matrix secretion and inhibition of cell apoptosis, thus producing the effect of preventing or treating osteoarthritis.

### CROSS REFERENCE TO RELATED APPLICATION

This disclosure claims the priority of Chinese Patent Application NO. 202010863064.X entitled Use of miRNA in preparation of a drug for preventing and treating osteoarthritis, an exosome highly expressing miRNA and use thereof filed with China National Intellectual Property Administration on Aug. 25, 2020, which is incorporated herein by reference in its entirety.

### TECHNICAL FIELD

The present disclosure relates to the technical field of miRNA and exosomes, in particular to the use of miR-155-5p in preventing and treating osteoarthritis, exosomes derived from synovial mesenchymal stem cells with high expression of miR-155-5p and use thereof.

### BACKGROUND

Osteoarthritis (OA) is one of the most common joint diseases and has become the main cause of disability in the elderly people. Studies have found that multiple factors such as trauma, abnormal mechanical load, lack of nutrition, and genetic predisposition can lead to occurrence of osteoarthritis. At present, there are still many difficulties to overcome in the treatment of osteoarthritis, because most drugs for the treatment of osteoarthritis can only relieve joint pain, but joint damage has not been improved.

The occurrence of osteoarthritis is mainly related to the decrease in the number of chondrocytes in the joint tissues, to the increase of cell apoptosis and to the metabolism disorder of extracellular matrix. From the perspective of the pathogenesis of osteoarthritis, exploring methods for treating osteoarthritis has always been the basis for researchers to explore new treatments and prevention methods.

### SUMMARY OF THE DISCLOSURE

In order to solve the above problems, the present disclosure provides the use of miRNA in preparation of drugs for preventing and treating osteoarthritis, the exosomes highly expressing miRNA and its use. The use of miR-155-5p provided by the present disclosure in preparation of drugs for preventing and treating osteoarthritis provides a new, more convenient and reliable solution for preventing and treating osteoarthritis.

In order to achieve the above objectives, the present disclosure provides the following technical solutions:

The present disclosure provides the use of miR-155-5p in preparation of drugs for preventing and/or treating osteoarthritis. The nucleotide sequence of miR-155-5p is set forth in SEQ ID NO.:1.

The present disclosure provides the use of miR-155-5p in preparation of osteoarthritis drugs that promote cell proliferation, migration, inhibit cell apoptosis and regulate the secretion of extracellular matrix. The nucleotide sequence of miR-155-5p is set forth in SEQ ID NO.:1.

The present disclosure provides the use of miR-155-5p in preparation of an osteoarthritis drug that promotes cell proliferation, migration, inhibits cell apoptosis, and regulates the secretion of extracellular matrix by targeting Runx2. The nuclear of miR-155-5p the nucleotide sequence is set forth in SEQ ID NO.: 1.

The present disclosure provides a synovial mesenchymal stem cell-derived exosome, which is characterized in that the synovial mesenchymal stem cell-derived exosome overexpresses miR-155-5p.

The present disclosure provides the use of the synovial mesenchymal stem cell-derived exosome that highly expresses miR-155-5p in preparation of drugs for preventing and/or treating osteoarthritis.

The use of miR-155-5p in the application in prevention and treatment of osteoarthritis provided in the present disclosure allows for improving miR-155-5p expression. By targeting miR-155-5p to Runx2, proliferation and migration of chondrocytes, secretion of extracellular matrix can be promoted and cell apoptosis can be inhibited. It can be seen from the examples that the exosome derived from synovial mesenchymal stem cells with high expression of miR-155-5p can reduce damage caused by osteoarthritis and promote cartilage regeneration.

### BRIEF DESCRIPTION OF THE DRAWINGS

FIG. 1 is a diagram of synovial mesenchymal stem cells (SMSC).

FIG. 2 is a diagram of purified exosomes, where the arrow points to the exosomes.

FIG. 3 is a diagram of exosomes that have been internalized by chondrocytes.

FIG. 4 is a diagram of miRNA-155-5p among others expressed in synovial tissue.

FIG. 5 is a diagram of miR-155-5p among others expressed in SMSC exosomes.

FIG. 6 is a diagram of SMSC-155-5p exosomes inhibiting apoptosis of chondrocytes.

FIG. 7 is a diagram of SMSC-155-5p exosomes alleviating degeneration of chondrocytes.

FIG. 8 is a diagram of cartilage cell migration and cartilage cell migration rate; wherein the left panel is a diagram of chondrocyte migration, and the right panel is a statistical diagram of the migration rate.

FIG. 9 is a diagram of CCK-8 test results.

FIG. 10 is a diagram of OARSI score.

FIG. 11 is a diagram of results for Western blot analysis of miR-155-5p inhibiting apoptosis.

FIG. 12 is a diagram of results for Western blot analysis of miR-155-5p increasing ECM secretion from OA chondrocytes.

### DETAILED DESCRIPTION OF THE EMBODIMENTS

The present disclosure provides the use of miR-155-5p in preparation of drugs for preventing and/or treating osteoarthritis. The nucleotide sequence of miR-155-5p is set forth in SEQ ID NO.: 1.

SEQ ID NO.: 1:
CTGTTAATGCTAATCGTGATAGGGGTTTTTGCCTCCAACTGACTCCTAC
ATATTAGCATTAACAG.

The present disclosure also provides the use of the miR-155-5p in preparation of osteoarthritis drugs that promote cell proliferation, migration, inhibit cell apoptosis and regulate the secretion of extracellular matrix. The nucleotide sequence of the miR-155-5p is set forth in SEQ ID NO.: 1.

The present disclosure further provides the miR-155-5p in preparation of osteoarthritis drugs that promotes cell proliferation and migration, inhibits cell apoptosis and regulates secretion of extracellular matrix by targeting Runx2. The nucleotide sequence of miR-155-5p is set forth in SEQ ID NO.: 1. Runx2 is responsible for the proliferation and differentiation of chondrocytes, miR-155-5p and Runx2 have binding sites, and miR-155-5p can targeted bind and negatively regulate the expression of Runx2, thereby preventing and/or treating osteoarthritis effect.

The present disclosure also provides a synovial mesenchymal stem cell-derived exosome, the miR-155-5p between high expressions of synovial mesenchymal stem cell-derived exosomes highly expressing miR-155-5p. In the present disclosure, synovial mesenchymal stem cell-derived exosome with high expression of miR-155-5p (SMSC-155-5p-Exos) of the present disclosure increases the expression of miRC-155-5p preferably in 67 folds compared with the expression of miR-155-5p by the synovial mesenchymal stem cell-derived exosome(SMSC-Exos). The expression of miR-155-5p in SMSC-155-5p-Exos chondrocytes treated with SMSC-155-5p-Exos increases by nearly 40 folds higher than the expression in chondrocytes without being treated with SMSC-155-5p-Exos.

In the present disclosure, method for preparation of the SMSC-155-5p-Exos is a conventional preparation method, preferably comprising: extracting synovial tissue from the cartilage in total knee arthroplasty patients having osteoarthritis (OA), removing the fat and part of connective tissue, cutting the synovial tissue into pieces, adding a DMEM culture medium containing collagenase II and 20% fetal bovine serum (FBS), digesting overnight in a CO₂ incubator; inoculating and suspending the cell precipitate in a 60 mm culture dish, changing the medium after 24 hours and removing non-adherent cells and changing the medium every 3 days; passaging the cells when the cells grow to 80% confluence; Culturing the cells in a DMEM culture medium containing 10% fetal bovine serum (FBS), 25 μg/ml ascorbic acid 2-phosphate and 1% penicillin streptomycin under the conditions of 37° C. and 5% carbon dioxide to obtain synovial mesenchymal stem cells (SMSC); Culturing SMSC at a concentration of 100 nM using the transfection reagent Lipofectamine® 2000 and transfecting the miR-155-5p mimic to obtain transfected SMSC. Separating and concentrating the transfected SMSC to obtain the SMSC-155-5p-Exos.

The present disclosure provides the use of the above mentioned synovial mesenchymal stem cell-derived exosome highly expressing miR-155-5p in preparation of a drug for prevention and treatment of osteoarthritis. SMSC-155-5p-Exos is convenient and reliable as a medicine for the treatment of OA. The overexpression of SMSC-155-5p-Exos can reduce the damage caused by osteoarthritis and promote cartilage regeneration, which can be used as a new type of means for treatment of OA.

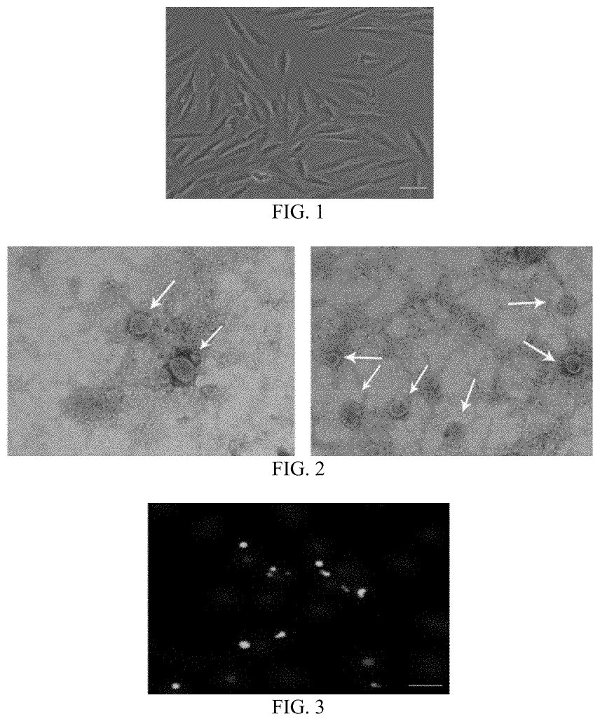

In the present disclosure, the synovial mesenchymal stem cells (SMSC) are shown in FIG. 1; the purified exosomes are shown in FIG. 2; and the exosomes that have been internalized by chondrocytes are shown in FIG. 3.

To further illustrate the present disclosure, the present disclosure will be described in detail in combination with accompanying drawings and embodiments of the present disclosure provides the use of miRNA in preparation of drugs for preventing and treating osteoarthritis, an exosome highly expressing miRNA and thereof, but they should not be construed as a limitation to the scope of protection of the present disclosure.

### Example 1

The cartilage tissue from the cartilage in total knee arthroplasty patients having osteoarthritis was extracted. The cartilage tissue was extracted with collagenase II and the primary chondrocytes were obtained. The chondrocytes were cultured in a DMEM culture medium containing 10% fetal bovine serum (FBS), 25 μg/ml ascorbic acid 2-phosphate and 1% penicillin streptomycin under the conditions of 37° C. and 5% carbon dioxide to obtain OA chondrocytes. The OA chondrocytes were cultured at a concentration of 100 nM using the transfection reagent Lipofectamine® 2000 and the miR-155-5p mimic was transfected to obtain transfected chondrocytes. The miR-155-5p mimic was transfected in SMSC which was separated and concentrated to obtain the SMSC-155-5p-Exos.

The method of separation and concentration comprised: culturing and transfecting chondrocytes with serum-free DMEM medium to obtain the culture supernatant; subjecting the culture supernatant to a first centrifugal treatment (centrifugal force 1000 r/min) to obtain a first supernatant; subjecting the first supernatant to a second centrifugal treatment (centrifugal force 3000 r/min) to obtain a second supernatant; subjecting the second supernatant to ultrafiltration and concentration treatment using a 100 kd ultrafiltration tube to obtain the ultrafiltrate; subjecting the ultrafiltrate to a third centrifugal treatment (centrifugal force is 10000 r/min) to obtain the third supernatant, and filtering and sterilizing the third supernatant with a 0.22 μm filter to obtain a concentrated solution; subjecting the concentrated solution to a fourth centrifugal treatment (centrifugal force 100000 r/min), and discarding the supernatant to obtain SMSC-155-5p-Exos.

### Comparative Example 1

Human synovial membrane-derived mesenchymal stem cells (SMSCs) were incubated in DMEM medium containing 10% FBS. After culturing in vitro for 3 days, the exosomes were separated and concentrated, and the method of separation and extraction of exosomes was the same as that in Example 1. The extracted exosomes(SMSC-Exos) derived from synovial mesenchymal stem cells have particles of similar size, with an average size of 100 nm and a modal density of 1.0-1.2 g/ml. Related markers of exosomes include CD63 and CD81.

### Application Example 1

Twenty specific pathogen-free (SPF) BALB/C mice were selected and randomly divided into 4 groups after subjected to 5 days of adaptation feeding:

Normal group: without cold water stimulation, 5 mice having 10 knee joints, n=10;

OA group; the mice were placed in 4° C. for 2 hours of cold stimulation, 2 times each day. Intra-articular injection of saline was conducted after 20 days of cold stimulation for consecutive 2 weeks and one time for each day. OA mouse model was created. Five mice had 10 knee joints, n=10.

OA+SMSC exosome group: an OA mouse model was created by using the same method as the OA group and the SMSC-Exos(30 μL; 10¹¹ exosomes/mL) provided in Comparative Example 1 was injected into the joint cavity for two consecutive weeks, with one time for each day. Five mice had 10 knees, n=10;

OA+SMSC-155-5p exosome group: an OA mouse model was created by using the same method as the OA group and the SMSC-155-5p-Exos (304; 10¹¹ exosome particles/mL) provided in Example 1 was injected into the joint cavity for consecutive 2 weeks, with one time for each day. Five mice had 10 knees, n=10.

In the OA group, the OA+SMSC exosome group and the OA+SMSC-155-5p exosome group, after injection of the normal saline or exosomes into the joint cavity of mice for 2 weeks, the joint tissues were taken for related indicator detection.

### Application Example 2

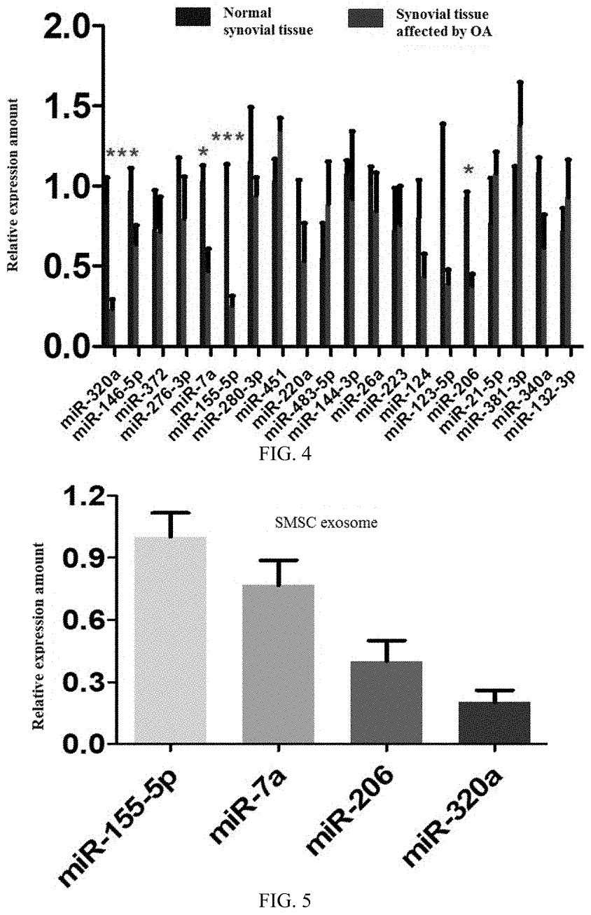

Synovial tissue and SMSC-exosomes were taken and extracted for total RNA according to the instructions by using TRIzol reagent (purchased from Invitrogen). Human microRNA qRT-PCR test kit was used to test for miRNAs by the cDNA reverse transcription and qRT-PCR, and the miRNAs includemiR-7a, miR-206, miR-320a, miR-155-5p etc. (GenScript; Nanjing). The sequences of miRNAs (SEQ ID NO.: 2-43) are shown in Table 1. RT-PCR was performed by using TransStart®Top Green qPCRSuperMix(Transgen Biotech), and the GAPDH was used as the internal reference standards of the result. The results are shown in FIG. 4 and FIG. 5, where FIG. 4 is a diagram showing the expression of miRNA-155-5p among others in synovial tissue, and FIG. 5 is a bar graph showing the relative expression amount of miR-155-5p among others in SMSC exosomes.

TABLE 1
Primer sequence for RT-PCR (SEQ ID NO.: 2-43)

| Gene | Upstream primer | Downstream primer |
|---|---|---|
| GAPDH | SEQ ID NO.: 2 aatcgccgtacccctacga | SEQ ID NO.: 3 gccctatatgagctcctgt |
| miR-320a | SEQ ID NO.: 4 tacccgttggctccaggt | SEQ ID NO.: 5 ccggtggttttgtgacatcc |
| miR-146-5p | SEQ ID NO.: 6 gctaagcttactggttag | SEQ ID NO.: 7 ccctcggtatttacgccggt |
| miR-372 | SEQ ID NO.: 8 gactaagcgcctttcgta | SEQ ID NO.: 9 aacgatggacgcccgtatcc |
| miR-276-3p | SEQ ID NO.: 10 atgcctcgttcatcaagaa | SEQ ID NO.: 11 tagtcccctaacacattaag |
| miR-7a | SEQ ID NO.: 12 attgccccccgcaggacct | SEQ ID NO.: 13 tgcgcttgaaacccgccagg |
| miR-155-5p | SEQ ID NO.: 14 aattttgacccgcaaggcc | SEQ ID NO.: 15 tccaacgtagctgtcctgctt |
| miR-280-3p | SEQ ID NO.: 16 aaaagttcccgccctgtata | SEQ ID NO.: 17 tgggctagcacccttcggact |
| miR-451 | SEQ ID NO.: 18 cccccgaggttgccgaaatt | SEQ ID NO.: 19 ttgaacaaattggtcccttac |
| | SEQ ID NO.: 20 ccctccctaaggttatttgca | |
| miR-220a | SEQ ID NO.: 22 gttccagcccggcacaatcg | SEQ ID NO.: 21 aatccggctcacccaattctg |
| miR-483-5p | SEQ ID NO.: 24 gacccgtggcttaactgcca | SEQ ID NO.: 23 taagacgctttcccggcgtgt |
| miR-144-3p | SEQ ID NO.: 26 taatacaggccgggtcaaga | SEQ ID NO.: 25 tgggtcctggccccggatttc |
| miR-26a | SEQ ID NO.: 28 gagactactgctgtgggtaag | SEQ ID NO.: 27 acggttcggtacccctatacg |
| miR-223 | | SEQ ID NO.: 29 cctttgttcttaacccagtga |
| miR-124 | SEQ ID NO.: 30 ctcccaacggtggcctcaatg | SEQ ID NO.: 31 tggccagaattccctgcagta |
| miR-123-5p | SEQ ID NO.: 32 tgggccacatcccggctgcga | SEQ ID NO.: 33 acccgtgaactggccagccta |
| miR-206 | SEQ ID NO.: 34 tgttgcacccccagagcattg | SEQ ID NO.: 35 aaagctccccggacgttagta |
| miR-21-5p | SEQ ID NO.: 36 agaccggggaattgtgaggcc | SEQ ID NO.: 37 tccttaaaggcaccctcctgg |
| miR-381-3p | | SEQ ID NO.: 39 cgtgccctgggcgtggtaccg |
| miR-340a | SEQ ID NO.: 38 ttgcggccgtcccttacgatc | SEQ ID NO.: 41 cttgcccaactcctccgtaatg |
| miR-132-3p | SEQ ID NO.: 40 gttaagtgcgttaagacccgt | SEQ ID NO.: 43 cggttgccttcaagcctgaac |
| | SEQ ID NO.: 42 tcggtaacccgtgcgtgatta | |

It can be seen from FIG. 4 and FIG. 5 that the expression level of miR-155-5p is significantly higher than that of miR-7a, miR-206, and miR-320a.

### Application Example 3

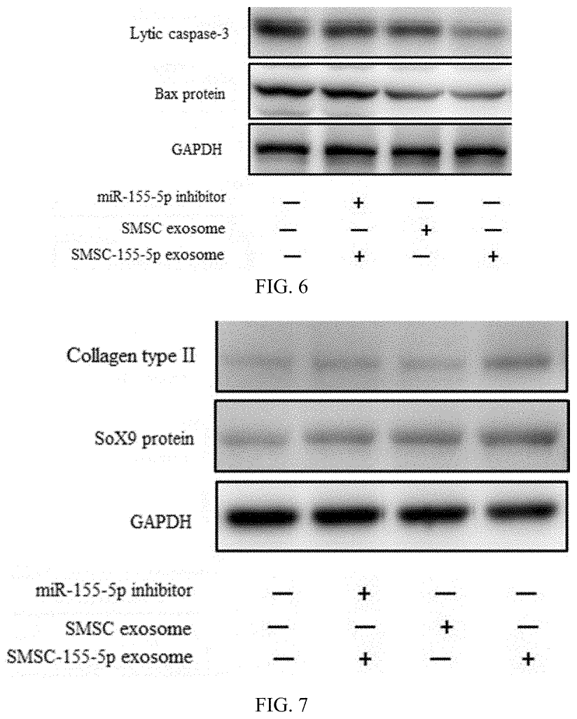

A lysis buffer containing 1% phosphatase inhibitor, 0.5% PMSF and 0.1% protease inhibitor (BC-WB-018; Biochannel, Nanjing) was used to extract protein from human chondrocytes. The boiled protein (30 μg) was added to SDS-PAGE and transferred to a PVDF membrane (Millipore, California, USA). Antibodies against Runx2 (ab76956), Caspase 3 (ab13847), Bax(ab32503), Bcl-2 (ab59348), Coll(ab34712), SOX9 (ab3967), and GAPDH (Santa Cruz Biotechnology, sc-137179). The primary antibody was incubated overnight at 4° C. at a dilution ratio of 1:1000 according to the manufacturer's instructions. Then, the membrane was incubated with the secondary antibody (ab97091) at room temperature for 2 hours, and the protein was detected with the supersignal West-Pico chemiluminescent substrate (Thermo-Fisher-Scientific). The test results are shown in FIG. 6 and FIG. 7. FIG. 6 is a diagram of SMSC-155-5p exosomes inhibiting chondrocyte apoptosis, and FIG. 7 is a diagram of SMSC-155-5p exosomes reducing chondrocyte degeneration. It can be seen from FIG. 6 and FIG. 7 that exosomes derived from synovial mesenchymal stem cells with high expression of miR-155-5p can significantly inhibit the apoptosis of chondrocytes and reduce the degeneration of chondrocytes.

### Application Example 4

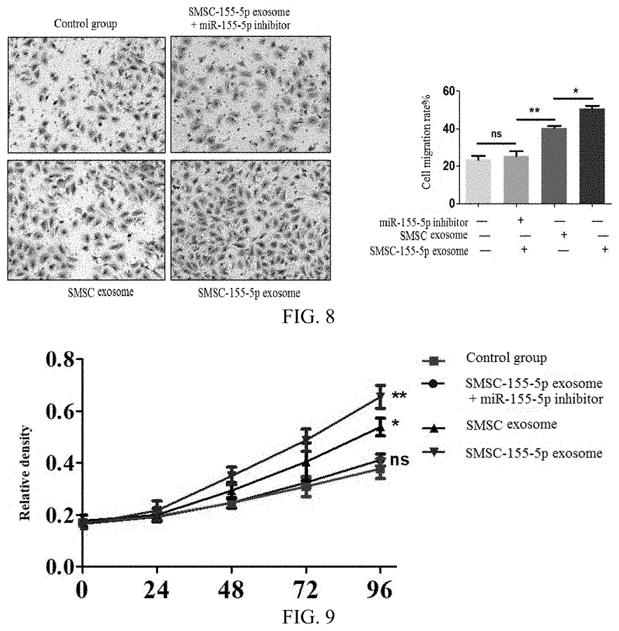

The Transwell system was used to detect the migration of chondrocytes. 5×10⁴ OA chondrocytes were placed in the upper chamber of a 24-well transwell plate (Corning, N.Y., USA). Then the control group (without addition), 0.5% FBS and 500 μL DMEM containing 1% PS; SMSC exosomes, 0.5% FBS and 500 μL DMEM containing 1% PS; SMSC-155-5p exosomes, 0.5% FBS and 5004 DMEM containing 1% PS; SMSC-155-5p exosomes+miR-155-5p inhibitor, 0.5% FBS and 5004 DMEM containing 1% PS were added in the lower chamber and cultured for 16 hours. The cells in the upper chamber were fixed with 4% paraformaldehyde for 20 minutes and stained with 0.5% hematoxylin-eosin for 10 min. After deleting the cells that had not migrated to the bottom surface, the cell migration rate of each well was calculated and evaluated by a double-blind method, and the results are shown in FIG. 8. The left side of FIG. 8 is a diagram of cartilage cell migration, and the right diagram is a statistical graph of migration rate. It can be seen from FIG. 8 that the SMSC-155-5p exosomes provided by the present disclosure can effectively promote the migration of chondrocytes.

### Application Example 5

The CCK-8 test kit was used to test the proliferation of human chondrocytes. The control group (without addition), SMSC exosomes, SMSC-155-5p exosomes, SMSC-155-5p exosomes+miR-155-5p inhibitors were used to stimulate human chondrocytes. After 6 hours, the transfected cells were inoculated in a 96-well plate and incubated at 37° C. in 5% CO₂ for a specified period of time. Each sample was divided into three equal parts for analysis. The cell viability was measured at 0 h, 24 h, 48 h, 72 h and 96 h. The optical density (OD) of each well was measured on a 450 nm multi-scan GO microplate reader (Thermo Fisher Scientific, Waltham, Mass., USA), and the detection results are shown in FIG. 9. FIG. 9 is a diagram of CCK-8 test results. It can be seen from FIG. 9 that SMSC-155-5p-Exos can effectively improve the survival rate of human chondrocytes.

### Application Example 6

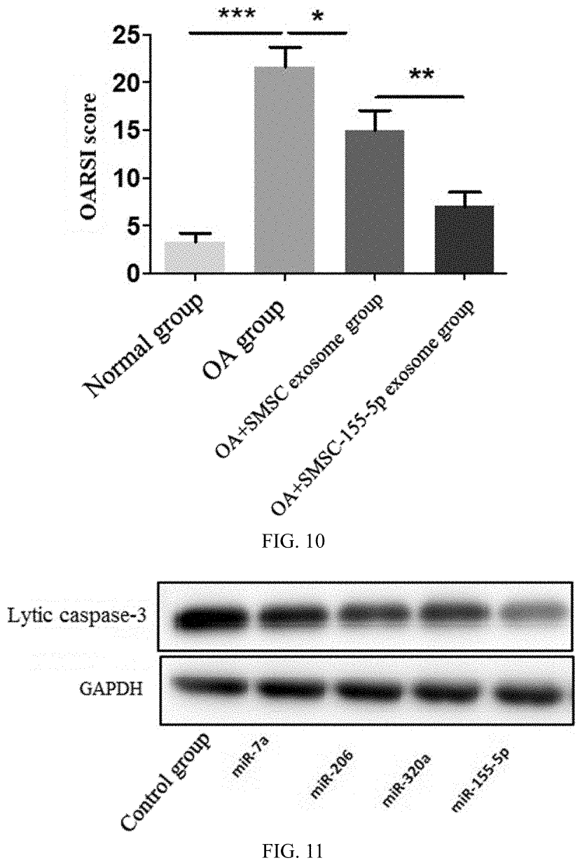

The cartilage tissue of mice in groups 1 to 4 in Application Example 1 were fixed overnight with 10% neutral formalin (Sigma), and decalcified with 30% (v/v) buffered formic acid. After decalcification, the samples were dehydrated and embedded in paraffin wax. The paraffin wax was serially sectioned in 5 μm thickness, and Coll cartilage matrix protein and P65 apoptotic protein were detected through immunohistochemical methods. The immunohistochemical photos were randomly selected, identified by 2 triple-blind pathologists, and scored using the OARSI scoring. The OARSI scoring results are shown in FIG. 10.

It can be seen from FIG. 10 that SMSC-155-5p-Exos has a better preventive effect on OA than SMSC-Exos does.

### Application Example 7

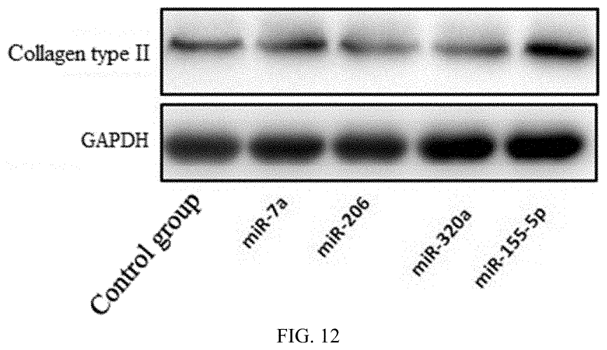

According to the preparation method provided in Example 1, OA chondrocytes were cultured with Lipofectamine® 2000 transfection reagent at a concentration of 100 nM and transfected with miR-7a mimics, miR-206 mimics and miR-320a mimics, respectively. The chondrocytes obtained after being transfected by the above-mentioned mimics, the blank control group and the chondrocytes in Example 1 were detected, and the changes in the degeneration of the chondrocytes were observed. The detection results are shown in FIG. 11 and FIG. 12. FIG. 11 is a diagram of the results for Western blot analysis of miR-155-5p inhibiting apoptosis, and FIG. 12 is a diagram of results for the Western blot analysis of miR-155-5p increasing ECM secretion from OA chondrocytes. It can be seen from FIGS. 11 and 12 that the technical effect of preventing cartilage cell degradation is achieved in Example 1.

It can be seen from the above application examples that the use of miRNA provided by the present disclosure in preparing drugs for preventing and treating osteoarthritis, exosomes with high miRNA expression and its use. Its use reduces osteoarthritis damage and promotes cartilage regeneration, and the technical effect of treatment and prevention of osteoarthritis is achieved.

Although the present disclosure has been disclosed as above in preferred embodiments, it is not intended to limit the present disclosure. Anyone familiar with this technology may make various changes and modifications without departing from the spirit and scope of the present disclosure. Therefore, the protection scope of the present disclosure should be defined by the claims.

What is claimed is:

1. A synovial mesenchymal stem-cell derived exosome comprising miR-155-5p.

2. The synovial mesenchymal stem cell-derived exosome of claim 1, wherein the miR-155-5p has the nucleotide sequence of SEQ ID NO: 1.

3. A method for treating osteoarthritis, comprising the step of administering a pharmaceutical composition to a patient with osteoarthritis, wherein the pharmaceutical composition comprises a synovial mesenchymal stem cell-derived exosome according to claim 1, and a pharmaceutically acceptable carrier therefor.

4. The method according to claim 3, wherein the synovial mesenchymal stem-cell derived exosome comprises miR-155-5p and the miR-155-5p has the nucleotide sequence of SEQ ID NO: 1.

5. A method for promoting chondrocyte proliferation and migration, inhibiting chondrocyte apoptosis and regulating secretion of extracellular matrix, comprising the step of administering a pharmaceutical composition according to claim 3 to a patient in need thereof.
